(19) United States
(12) Patent Application Publication
(10) Pub. No.: US 2022/0175412 A1
(43) Pub. Date: Jun. 9, 2022

(54) SURGICAL INSTRUMENT WITH FLEXIBLE END EFFECTOR

(71) Applicant: GYRUS ACMI, INC. D/B/A OLYMPUS SURGICAL TECHNOLOGIES AMERICA, Westborough, MA (US)

(72) Inventor: Nikhil M. Murdeshwar, Maple Grove, MN (US)

(21) Appl. No.: 17/457,316

(22) Filed: Dec. 2, 2021

Related U.S. Application Data
(60) Provisional application No. 63/121,562, filed on Dec. 4, 2020.
(60) Provisional application No. 63/270,502, filed on Oct. 21, 2021.

(51) Int. Cl. A61B 17/3209; A61B 17/42

(52) U.S. Cl. CPC A61B 17/3209; A61B 2017/00017; A61B 2017/4216; A61B 17/4241

(57) ABSTRACT

A surgical instrument for removing biological tissue during a surgical procedure comprises an elongate shaft and an end effector connected to the elongate shaft, the end effector including an edge configured to remove tissue, wherein curvature of one or both of the elongate shaft and end effector is adjustable to position the edge along a tissue surface when the curvature of the one or both of the elongate shaft and end effector is adjusted. A method of incising target tissue from an anatomic wall comprises inserting a shaft of a surgical instrument into an anatomic chamber of a patient, positioning a tissue-removal device connected to the shaft proximate the target tissue, deflecting an axis of the tissue-removal device relative to a central axis of the shaft, and shaving a surface of the anatomic wall by moving the tissue-removal device in a deflected state along the target tissue.

### CROSS-REFERENCE TO RELATED APPLICATIONS

This application claims the benefit of priority to U.S. Provisional Patent Application No. 63/121,562, filed Dec. 4, 2020; and U.S. Provisional Patent Application Ser. No. 63/270,502, filed Oct. 21, 2021, the contents of which are incorporated herein by reference in their entireties.

### TECHNICAL FIELD

This document pertains generally, but not by way of limitation, to surgical instruments and methods that include surgical tooling and instrumentation that can be used for open and laparoscopic surgical procedures. More specifically, but not by way of limitation, the present application relates to systems and methods for preventing, treating, removing and monitoring diseased or potentially diseased tissue.

### BACKGROUND

Many surgical procedures involve the treatment or removal of target tissue, e.g., diseased, potentially diseased or otherwise unwanted tissue, located inside of a patient. As such, these procedures require access to the internal anatomy of the patient via an open procedure or through a smaller opening in minimally invasive (e.g., laparoscopic) procedures. In either case, the surgeon is required to manipulate surgical instruments within tight confines of the internal anatomy to identify target tissue that is surrounded by other tissue, and diagnose and remove or otherwise treat the target tissue. Many surgical instruments exist to excise or treat target tissue that might be diseased. For example, surgical tooling, such as ablation devices, cauterizing devices and cutting forceps, can be inserted into a patient and manipulated from a handpiece that extends from the surgical tooling via a shaft.

Endometriosis is a condition in which endometrium tissue that typically lines the inside of a uterus spreads to other places in the abdomen. The condition can be particularly painful as the endometrium tissue outside the uterus continues to behave in the manner of endometrium within the uterus during the menstrual cycle by thickening, breaking-down and bleeding. Treatment for endometriosis involves removing the endometrium tissue outside the uterus. As such, it is desirable to identify the endometrium tissue such that other healthy tissue in the abdomen is not unnecessarily removed and to ensure that all of the extra-uterine endometrium tissue is identified to eliminate the endometriosis and its symptoms and the need for a follow-up procedure. Identification of endometrium tissue can be facilitated by the use of dyes whereby a patient ingests a dye that can metabolize to or otherwise be absorbed by the endometrium tissue. The dye can then be energized with light of a particular wavelength to illuminate the tissue containing the dye. However, use of dyes requires light be introduced into the surgical site, which typically requires use of an additional instrument. In any event, the identification and treatment of endometriosis can be difficult.

### OVERVIEW

The present inventors have recognized, among other things, that problems to be solved in performing medical procedures to remove diseased tissue include the difficulty in accurately locating and assessing diseased tissue, such as a lesion, cancer cells, or endometriotic tissue, treating the tissue, preventing the formation of follow-on conditions, and post-operative monitoring of treated tissue.

The present inventors have recognized, among other things, that problems to be solved in performing medical procedures include the difficulty in removing diseased tissue from a small cavity within an anatomical area of a patient. In particular, laparoscopic procedures involve inserting a laparoscope tube into the patient to allow a camera to view a surgical site. Other instruments used to perform the procedure on the target tissue viewed by the camera are inserted into the tube. Thus, the end effector, e.g., the working tool or instrument used to remove the diseased tissue, can be configured to be inserted through a lumen having a small diameter. It can, therefore, be difficult to orient the end effector of the working tool relative to the diseased tissue so as to, for example, remove the diseased tissue, e.g., superficial lesions, flush with the surface of the non-diseased tissue without removing excess tissue and risking organ damage.

The present subject matter can provide solutions to this problem and other problems, such as by providing medical devices, such as laparoscopy systems, that include flexible end effectors or tissue-removal devices that can be used to remove diseased or unwanted tissue. The flexible tissue-removal devices can be resilient and can be deflected or bent to lie flush or nearly flush with anatomical walls. The end effectors can be self-bending or bent with the use of an additional device or mechanism.

The present inventors have recognized that problems to be solved in performing medical procedures include the ability to properly identify target tissue for removal. For example, it can be difficult to visualize the anatomy for lesions within the patient through the laparoscopy equipment. Thus, it can be difficult to estimate the appropriate depth to which the resection can be performed to remove the lesion.

The present subject matter can provide solutions to this problem and other problems, such as by providing a surgical system, that can provide imaging to a surgeon to view lesions and other diseased anatomy within a patient. The surgical system can comprise a viewing device, such as a set of glasses, goggles, and/or a heads-up display, that can communicate with a reusable camera module that can be coupled to an elongate surgical instrument such as a disposable laparoscopy shaft. The viewing device can comprise a device configured to receive and display information form the camera module for the surgeon to view.

The present inventors have recognized that problems to be solved in performing diseased tissue removal procedures include the formation of post-operative adhesions.

The present subject matter can provide solutions to this problem and other problems, such as by providing medical devices, such as laparoscopy systems, that include means for applying adhesion suppressants to the site of tissue removal.

The present inventors have recognized that problems to be solved in performing medical procedures include the inability to monitor post-operative outcomes and, more specifically, the difficulty in treating recalcitrant patients following surgery.

The present subject matter can provide solutions to this problem and other problems, such as by providing implantable monitoring devices that can provide post-operative feedback. The monitoring devices can be implanted during a tissue-diagnosing or tissue-removal procedure. The monitoring devices can be left in the patient after the procedure to provide post-operative feedback to a physician. In examples, the monitoring devices can provide tactile stimulation of an internal anatomical point and/or monitor for inflammation, tissue growth and the like.

In an example, a surgical instrument for removing biological tissue during a surgical procedure can comprise an elongate shaft and an end effector connected to a distal portion of the elongate shaft, the end effector including an edge configured to remove tissue, wherein curvature of one or both of the elongate shaft and end effector is adjustable to position the edge along a tissue surface when the curvature of the one or both of the elongate shaft and end effector is adjusted.

In another example, a surgical instrument can comprise an elongate shaft extending along a central axis between a proximal end portion and a distal end portion, a control device connected to the proximal end portion, and a tissue-removal device comprising a flexible body connected to the distal end portion along a trajectory and a tissue removal feature on the flexible body, wherein a user of the surgical instrument can alter the trajectory of the flexible body by changing curvature of the flexible body.

In an additional example, a method of incising target tissue from an anatomic wall can comprise inserting a shaft of a surgical instrument into an anatomic chamber of a patient, positioning a tissue-removal device connected to the shaft proximate the target tissue, deflecting an axis of the tissue-removal device relative to a central axis of the shaft, and shaving a surface of the anatomic wall by moving the tissue-removal device in a deflected state along the target tissue.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1A is a schematic illustration of a surgical instrument having a flexible tissue-removal device in an undeflected state proximate diseased tissue.

FIG. 1B is a schematic illustration of the surgical instrument of FIG. 1A with the flexible tissue-removal device in a deflected state against resected tissue.

FIG. 2A is a schematic illustration of a surgical instrument having a pre-flexed tissue-removal device retracted into a laparoscope in a straightened state.

FIG. 2B is a schematic illustration of the surgical instrument of FIG. 2A with the pre-flexed tissue-removal device extended from the laparoscope in a curved state.

FIG. 3A is a schematic illustration of a surgical instrument having a flexible tissue-removal device retracted into a laparoscope in a straightened state alongside a pre-flexed brace.

FIG. 3B is a schematic illustration of the surgical instrument of FIG. 3A with the flexible tissue-removal device extended from the laparoscope and bent to a curved state by the pre-flexed brace.

FIG. 4A is a schematic illustration of a surgical instrument having a pre-flexed tissue-removal device extended from a laparoscope in a curved state with a brace retracted into the laparoscope.

FIG. 4B is a schematic illustration of the surgical instrument of FIG. 4A with the pre-flexed tissue-removal device extended from the laparoscope and straightened by the brace.

FIG. 5A is a schematic illustration of a surgical instrument having a flexible tissue-removal device extended from a laparoscope in an undeflected state with a steerable brace attached to the flexible tissue-removal device.

FIG. 5B is a schematic illustration of the surgical instrument of FIG. 5A with the steerable brace deflected to curve the flexible tissue-removal device a first amount.

FIG. 5C is a schematic illustration of the surgical instrument of FIG. 5A with the steerable brace deflected to curve the flexible tissue-removal device a second amount.

FIG. 6 is schematic view of a surgical system comprising a surgical headset and a disposable instrument shaft having a reusable camera module in communication with the surgical headset.

FIG. 7 is a diagram of a laparoscope device including a suppressant delivery system configured to deliver suppressant material to a uterus.

FIG. 8A is a schematic illustration of a physician holding a hand-held interrogation device and a patient shown behind an x-ray machine.

FIG. 8B is a schematic illustration of an x-ray image taken from the x-ray machine of FIG. 8A to show in-situ communication devices configured to communicate with the hand-held interrogation device.

FIGS. 9A and 9B are schematic illustrations of a surgical instrument comprising a flexible electrically conductive cutting device, which can be configured to bend similarly as is described with reference to the cutting instrument of FIGS. 1A-5C.

FIG. 10A is a schematic illustration of a tissue retrieval device comprising a scraping device and a spring-loaded actuator in a retracted state, which can be configured to bend similarly as is described with reference to the cutting instrument of FIGS. 1A-5C.

FIG. 10B is a schematic illustration of the tissue retrieval device of FIG. 10A with the spring-loaded actuator in a deployed state.

FIG. 11A is a top cross-sectional view of the tissue retrieval device of FIG. 10B taken along plane 11A-11A to show container space for the storage of collected matter.

FIG. 11B is a side cross-sectional view of the tissue retrieval device of FIG. 10A taken along plane 11B-11B to show a blade edge for the slicing of collected matter.

FIG. 11C is a side cross-sectional view of the tissue retrieval device of FIG. 10B taken along plane 11C-11C to show a sleeve positioned around the actuator and the container.

FIG. 12A is a schematic illustration of a tissue retrieval device comprising a boring device and an inflatable actuator in a collapsed state, which can be configured to bend similarly as is described with reference to the cutting instrument of FIGS. 1A-5C.

FIG. 12B is a schematic illustration of the tissue retrieval device of FIG. 12A with the inflatable actuator in an expanded state.

FIG. 13 is a schematic representation of an imaging and control system comprising a control unit connected to a scope.

FIG. 14 is schematic diagram of the imaging and control system of FIG. 13 connected to the scope.

FIG. 15A is an end view of a camera module including optical and functional components suitable for use with the scope of FIGS. 13 and 14.

FIG. 15B is a cross-sectional view taken along the plane 15B-15B of FIG. 15A showing components of the camera module.

FIG. 16 is a schematic illustration of a surgical instrument of the present disclosure comprising an elongate shaft, a tissue-removal device and an actuator device.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

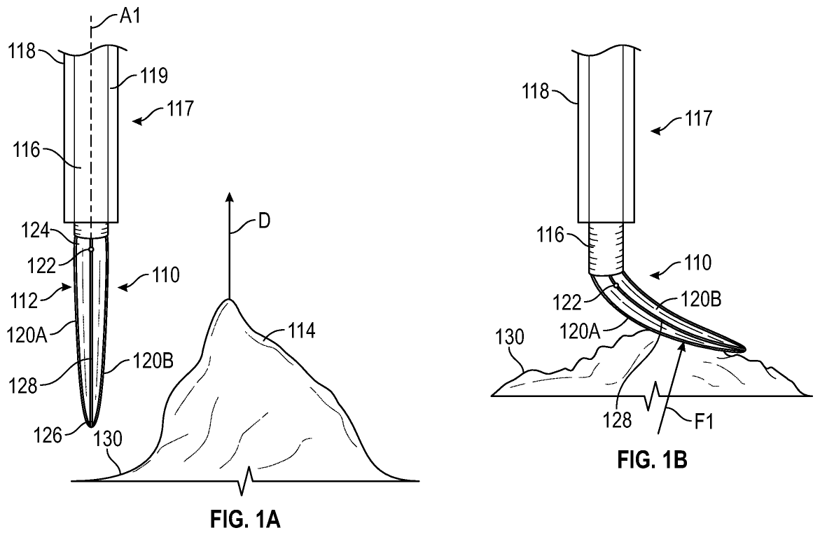

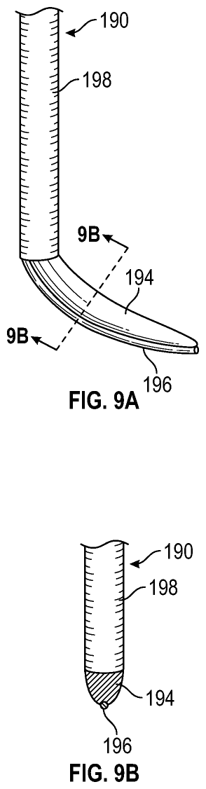

FIGS. 1A-5B show tissue-removal devices, such as flexible jaws, that can be bent at an angle, e.g., a right angle, to a shaft of an insertion device. FIGS. 9A and 9B show tissue-removal devices, such as a flexible electric cutting device, that can be bent at an angle, e.g., a right angle, to a shaft of an insertion device. The tissue-removal devices of the present disclosure can include tissue-removing edges that can be deformed to fit against or conform to surfaces of tissue to, for example, facilitate flush removal of tissue. With conventional tissue-removal devices, it can be difficult or impossible to bend the tissue-removing edges, such as orthogonal to the plane of FIG. 1A. The tissue-removal devices can comprise other devices for removing tissue, such as forceps, slicing devices, sawing devices, cutting devices, boring devices, augers and the like. Additionally, the tissue-removal devices of the present application can include rotatable devices, such as grinders or abraders, ablation devices, blades augmented with radio frequency (RF) energy. The illustrated examples of FIGS. 1A-5B show pivotable, e.g., hinged jaws, although other scissor type mechanisms can be used. In additional examples, the flexible tissue-removal devices of the present disclosure can deform in shape in other ways than bending, such as by expanding in an inflatable manner to become bulbous to match a tissue profile or expanding in an according manner to become bulbous or shaped to match a tissue profile. The tissue removal devices can facilitate clean resection, parallel to, or nearly parallel to or otherwise favorably oriented relative to the duct wall of the organ, the tissue surface, without risking removal of excess tissue from the under layers. The radius of curvature of the tissue removal devices can be fixed, varied or controlled depending on the particular configuration. The flexible tissue removal devices can spare organ damage when resecting lesions from bowel, liver, bladder, ureter, uterus, etc. The tissue removal devices can be made of shaped stainless steel, polymers, Nitinol and other shape memory alloys and other materials.

In examples, the tissue collector devices of the present disclosure can be controlled from a proximal end portion of an insertion shaft to control a distally located end effector, such as the tissue collector. The end effector can be adjustable by a user or operator from the proximal end to adjust the size and/or diameter, stiffness and/or conformable shape of the tissue collector device/end effector. A shape changing feature can comprise a hollow body and can inflate like a balloon, shorten via a pull wire, etc. In examples, the end effector can be hollow, and one or more holes can be provided in the end effector in communication with one or more delivery channels. The holes can have different diameters or sizes and can be arranged in different patterns to allow for delivery of one or more substances, such as irrigation to clear debris or injection of media like drugs, dyes, etc.

FIG. 1A is a schematic illustration of surgical instrument 110 having flexible tissue-removal device 112 in an undeflected state proximate target tissue 114. FIG. 1B is a schematic illustration of surgical instrument 110 of FIG. 1A with flexible tissue-removal device 112 in a deflected state against resected tissue. In FIG. 1A, the cutting, slicing or shaving trajectory of tissue removal device 112 is aligned with central axis A1 of shaft 116. In FIG. 1B, the cutting trajectory of tissue removal device 112 is deflected or curved away from central axis A1 of shaft 116. FIGS. 1A and 1B are discussed concurrently. In various examples, tissue-removal device 112 can include tissue-removing edges, either on outer edges or edges on an inner window, that can be deformed to fit against or conform to surfaces of tissue to, for example, facilitate flush removal of tissue with an anatomic wall such that scraping or reciprocation of device 112 can remove tissue in the deformed state.

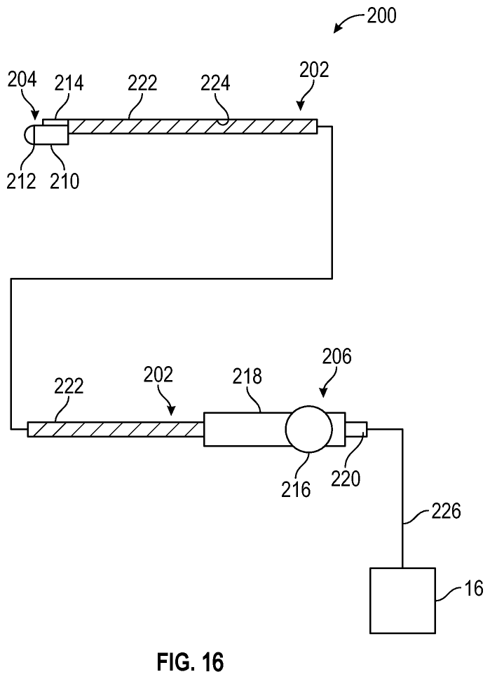

Surgical instrument 110 can comprise an example of surgical instrument 200 of FIG. 16. Surgical instrument 110 can comprise shaft 116 from which flexible tissue-removal device 112 can extend. Flexible tissue-removal device 112 can comprise an end effector for surgical instrument 110 that can be used to obtain tissue from a patient. Surgical instrument 110 can be used with scope 117. Surgical instrument 110 can extend along central axis A1 within scope 117. Thus, scope 117 can be substantially coaxial with or follow the same axial path as shaft 116 of surgical instrument 110. Tissue-removal device 112 can extend from shaft 116 along central axis A1 when tissue-removal device 112 is not deflected as shown in FIG. 1A. However, as shown in FIG. 1B, tissue-removal device 112 can be deflected by engagement with tissue 114 to cause curvature of tissue-removal device 112 away from central axis A1. As discussed herein, deflection of tissue-removal device 112 away from central axis A1 can be achieved by a variety of means and mechanism, such as manual deflection, pre-curved or biased elements, steering devices, mechanical actuators and inflatable actuators, as well as others.

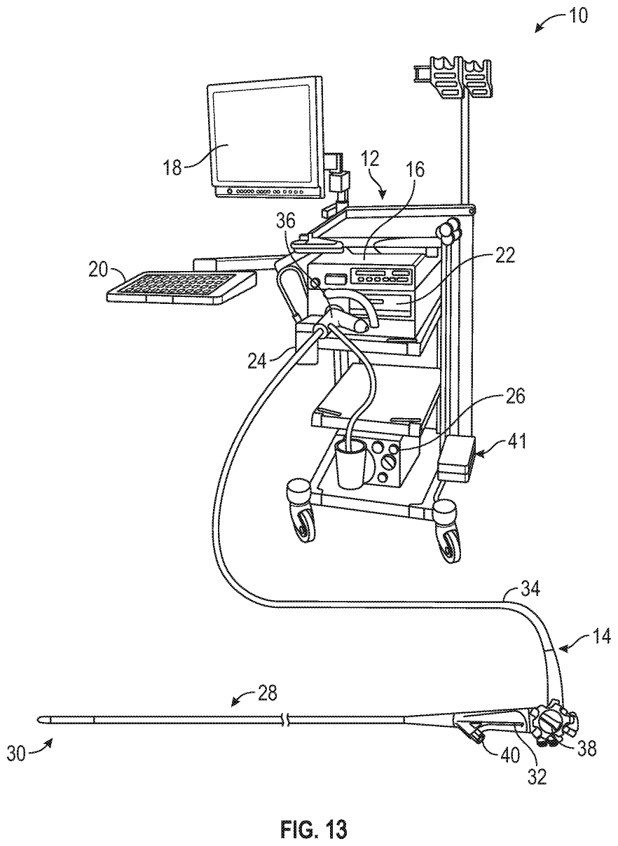

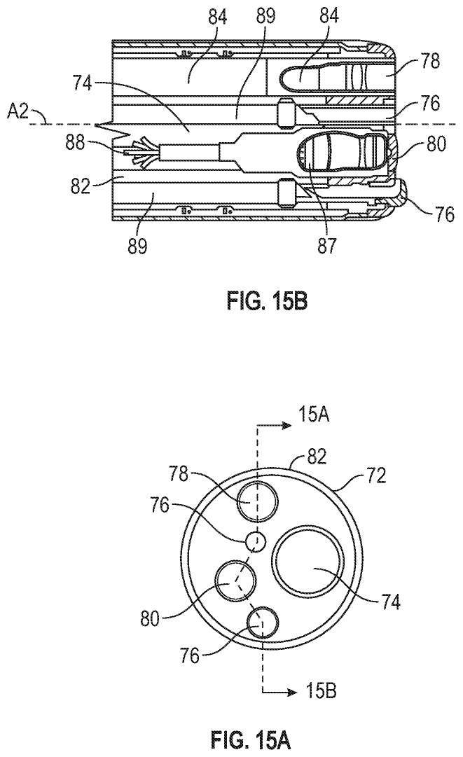

Scope 117 can comprise any suitable delivery device, such as an endoscope, laparoscope or a duodenoscope. In examples, scope 117 can comprise scope 14 of FIGS. 13-15B. Scope 117 can comprise shaft 118 having lumen 119 that can comprise a working channel, similar to therapy unit 74 of FIG. 15A, to receive various instruments such as surgical instrument 110. In additional examples, shaft 118 can comprise a component of tissue-removal device 112 and can be used independently of a scope or used in conjunction with a scope. In examples, shaft 118 can comprise shaft 222 of FIG. 16 and shaft 116 can be extended therefrom via operation of controller 206 by a user.

Shaft 116 can comprise a flexible and rigid body that supports tissue-removal device 112. Shaft 116 can include internal lumens or passages to connect tissue-removal device 112 to a proximate end of shaft 116 where operator controls can be located. Tissue-removal device 112 can comprise a cutting device having first component 120A and second component 120B. connected at hinge 122. Tissue-removal device 112 can have base 124 at shaft 116 and distal tip 26. In examples, tissue-removal device 112 can be configured as a scissors or forceps such that first component 120A and second component 120B form jaws. However, as discussed herein, tissue-removal devices can comprise scraping, sawing, slicing, cauterizing, ablating devices and the like. Inner opposing edges of first component 120A and second component 120B can be engaged along interface 128 configured to spread apart to receive tissue, such as by pivoting at hinge 122. Edges of first component 120A and second component 120B can be sharpened at interface 128 to form blades. In other examples, first and second components 120A and 120B can be serrated or include saw teeth, or a mixture of blades, serrations and teeth. In yet other examples, tissue-removal device 112 can comprise a tissue slicing device wherein first and second components 120A and 120B are connected as a single sheet or piece and a cutting edge can be located in place of interface 128. In such a configuration, the tissue-removal device 112 can be configured to shave or scrape tissue via rotation, similar to examples described below. In examples, first component 120A and second component 120B can be portions of a single sheet or piece of material and one or both other edges (edges opposite interface 128 in FIG. 1A that define an outer perimeter of tissue-removal device 112) of first component 120A and 120B can be sharpened.

First component 120A and second component 120B can be configured to be flexed, such as via the application of pressure or force from shaft 116 to engage tissue-removal device 112 with another body, such as tissue 114. In examples, first component 120A and second component 120B can be rigid so as to retain their own shape when not subject to external forces, but can be deflected when subject to force F1 as explained above. First component 120A and second component 120B can be resilient so as to return to their undeflected state after external forces, such as force F1, are removed.

In the configuration of FIG. 1A, central axis A1 of shaft 116 can align with interface 128 in an undeflected state of tissue-removal device 112. However, in the configuration of FIG. 1B, interface 128 can be curved relative to central axis A1 by the application of force F1 from tissue 114. Force F1 can be a force suitable for deflecting first component 120A and second component 120B without undue exertion from a user, with minimizing risk of puncturing through anatomy wall 130 and below a level where stresses in strains are generated in first component 120A and second component 120B that might give rise to permanent bending or breaking. In examples, first component 120A and second component 120B can be configured as thin, sheet metal components wherein the thickness into the plane of FIG. 1A is much smaller than the length along interface 128 or the transverse width from interface 128. First component 120A and second component 120B can additionally be fabricated from other materials, such as plastics, polymers, glass and ceramics. As such, first component 120A and second component 120B can be configured to flex into and out of the plane of FIG. 1A. However, tissue-removal device 112 can be flexed in other directions, as shown in FIG. 1B. In examples, first component 120A and second component 120B can be approximately 0.23 mm (0.009 inches) thick. In examples, first component 120A and second component 120B can be thicker near shaft 116 to provide more rigidity and thinner near distal tip 126 to provide more flexibility. Distal tip 126 can be sharp to facilitate piercing of tissue or can be blunt to facilitate sliding of tissue-removal device 112 along tissue for positioning of first component 120A and second component 120B proximate tissue 114.

As shown in FIG. 1A, tissue 114 can project from anatomy wall 130 in direction D. With conventional rigid cutting devices, it can be difficult to orient the device relative to tissue 114, e.g., parallel to anatomy wall 130 or otherwise transverse to direction D. However, with the devices of the present disclosure, interface 124 between first component 120 and second component 120B can be flexed to be tangent or oblique to direction D to allow tissue 114 to be more readily positioned between first component 120A and second component 120B. As such, tissue 114 can be removed close to anatomy wall 130 with a reduced risk of cutting too deep into anatomy wall 130 or puncturing through anatomy wall 130.

Example methods can include pushing tissue-removal device 112 against anatomy wall 130 away from tissue 114 in order to deflect tip 126 and the cutting trajectory of first component 120A and second component 120B away from central axis A1, opening first component 120A and second component 120B along engagement 128 by pivoting at hinge 122, sliding first component 120A and second component 120B into engagement with tissue 114, and then closing first component 120A and second component 120B along engagement 128 by pivoting at hinge 122 to incise or resect tissue 114.

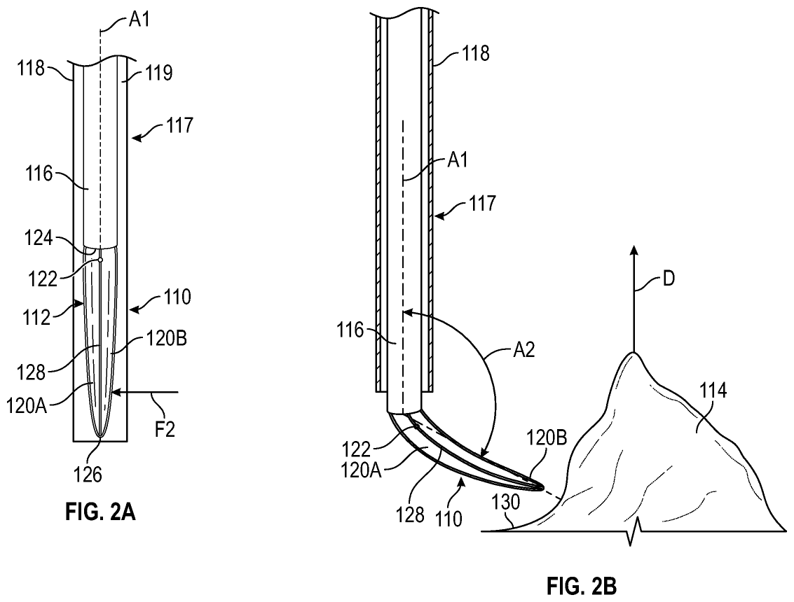

FIGS. 2A and 2B show an example of surgical instrument 110 wherein tissue-removal device 112 can be pre-curved relative to central axis A1 of shaft 116. In additional examples, a distal end portion of shaft 116 can be pre-curved relative to central axis A1 of a proximal portion of shaft 116 or pre-curved relative to shaft 118. As shown in FIG. 2B, first component 120A and second component 20B of tissue-removal device 112 can be curved without the application of force or engagement with tissue 114. However, first component 120A and second component 120B can be flexible so as to allow retraction of tissue-removal device 112 into scope 117. Thus, as shown in FIG. 2A, force F2 can be applied to tissue-removal device 112 via shaft 118 to straighten tissue-removal device 112. In examples, first component 120A and second component 120B can be fabricated of shape memory alloy, such as Nitinol or Nickel titanium. In examples, first component 120A and second component 120B can be fabricated of stainless steel that is deformed into a curved shape. In additional examples, first component 120A and second component 120B can be fabricated from plastic materials, with or without internal or external reinforcements that can aid in pre-pending, such as pre-curved metallic wires that can extend along first component 120A and second component 120B. First component 120A and second component 120B can be configured to curve such that distal tip 126 is positioned relative to shaft 118 at angle AA. In examples, angle AA can be in the range of approximately ninety degrees to approximately one-hundred-sixty degrees. In examples angle AA can be in the range of approximately one-hundred-ten degrees to approximately one-hundred-thirty degrees. However, tissue-removal device 112 can be configured to pre-bend at other values of angle AA. Additionally, in the examples of FIGS. 2A and 2B, first component 120A and second component 120B can be flexible as explained with reference to FIGS. 1A and 1B such that angle AA can be reduced by pushing tissue-removal device 112 into tissue anatomy wall 130 or can be increased by pulling on tissue. In examples, surgical instrument 110 can be rotated about central axis A1 within lumen 119 such that tissue-removal device 112 can be angled in different directions relative to scope 117.

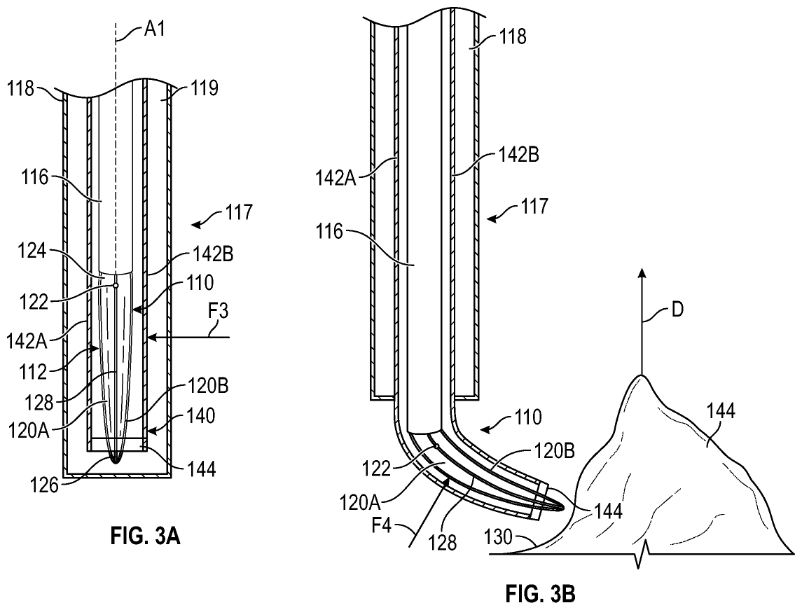

FIGS. 3A and 3B show an example of surgical instrument 110 wherein tissue-removal device 112 can be curved relative to central axis A1 of shaft 116 via a separate mechanism, such as before engaging, or without having to engage, tissue 114. In examples, the separate mechanism for curving tissue-removal device 112 can comprise brace 140. Brace 140 can comprise extensions 142A and 142B and guide 144.

Thus, first component 120A and second component 120B can be configured to be straight at rest, as shown in FIG. 3A, and then flexed by advancing pre-curved brace 140 that can ride along, e.g., on top of, first component 120A and second component 120B. First component 120A and second component 120B can be configured to be curved by brace 140 such that distal tip 126 is positioned relative to shaft 118 at angle AA, as explained with reference to FIG. 2B. Thus, scope 117 can include features, such as shaft 118, for maintaining brace 140 in a straightened state. As shown in FIG. 3A, force F3 can be applied by shaft 118 to straighten brace 140 inside shaft 118. Force F3 can be a force suitable for deflecting extensions 142A and 142B without undue effects on shaft 118 and below a level where stresses in strains are generated in extensions 142A and 142B that might give rise to permanent bending or breaking. Thus, tissue-removal device 112 can be advanced from shaft 118 without brace 140 to be in a straight configuration. As shown in FIG. 3B, force F4 can be applied by brace 140 to tissue-removal device 112 when both brace 140 and tissue-removal device 112 are outside shaft 118 to curve tissue-removal device 112. Force F4 can be a force suitable for deflecting first component 120A and second component 120B and below a level where stresses in strains are generated in first component 120A and second component 120B that might give rise to permanent bending or breaking.

Extensions 142A and 142B can comprise elongate bodies, such as wires, cables that can be extended from and retracted into scope 117. Extensions 142A and 142B can comprise pre-curved bodies, as has been described above. In examples, extensions 142A and 142B can be rigid so as to retain their own shape when not subject to external forces, but can be deflected when subject to force F3 as explained above. Extensions 142A and 142B can be resilient so as to return to their undeflected state after external forces such, as force F3, are removed. In examples, extensions 142A and 142B can be fabricated of shape memory alloy, such as Nitinol or Nickel titanium. In examples, extensions 142A and 142B can be fabricated of stainless steel that is deformed into a curved shape. Guide 144 can comprise a body having a slot to receive tissue-removal device 112. For example, guide 144 can comprise a four-sided box having two sides connected to extensions 142A and 142B, respectively, and two other sides that lay flat against first component 120A and second component 120B, thereby bounding tissue-removal device 112. As such, first component 120A and second component 120B can still be permitted to pivot at hinge 122. Extensions 142A and 142B can be pre-curved when free of an external force, such as a force from being engaged with tissue 114. Thus, guide 144 can impart deflection or curvature to first component 120A and second component 120B. In examples, guide 144 need not extend close to tip 126, but can be positioned proximate base 124 to impart deflection or curvature to first component 120A and second component 120B. In examples, guide 144 can comprise a body or plate that is located on a single side of tissue-removal device 112 to push first component 120A and second component 120B in a single direction. Guide 144 can include magnetic capabilities to attract first component 120A and second component 120B. Guide 144 can be attached to tissue-removal device 112 via a slotted connection that can permit guide 144 to slide axially along tissue-removal device 112, that can be retracted to allow first component 120A and second component 120B to be at their natural state and advanced to allow first component 120A and second component 120B to be deflected or curved.

In examples, surgical instrument 110 and brace 140 can be independently rotated about central axis A1 within lumen 119 such that tissue-removal device 112 can be angled in different directions relative to scope 117. In examples, guide 144 can comprise an arcuate plate rotatable about central axis A1 via extensions 142A and 142B to push tissue-removal device 112 in selected radial directions.

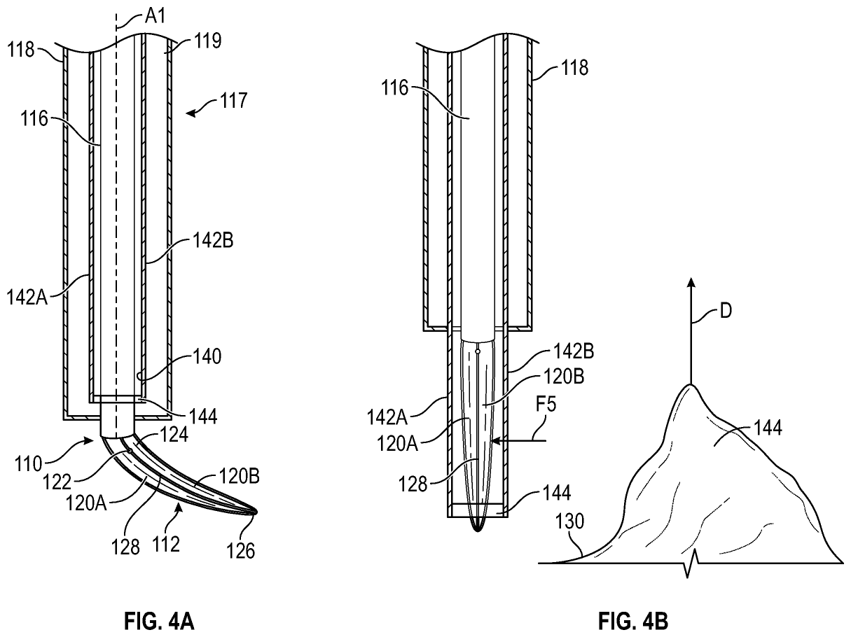

FIGS. 4A and 4B show an example of surgical instrument 110 wherein tissue-removal device 112 can be pre-curved relative to central axis A1 of shaft 116, such as before engaging tissue 114, and then straightened via a separate mechanism. In examples, the separate mechanism can comprise brace 140. Thus, first component 120A and second component 120B can be configured to be curved at rest, as shown in FIG. 4A, and then straightened by advancing straight brace 140 that can ride along, e.g., on top of, first component 120A and second component 120B to apply force F5 (FIG. 4B). Thus, surgical instrument 110 can be configured similarly as that described with reference to FIGS. 2A and 2B. However, brace 140 can be configured with extensions 142A and 142B similar to those described with reference to FIGS. 3A and 3B, but without the pre-curve.

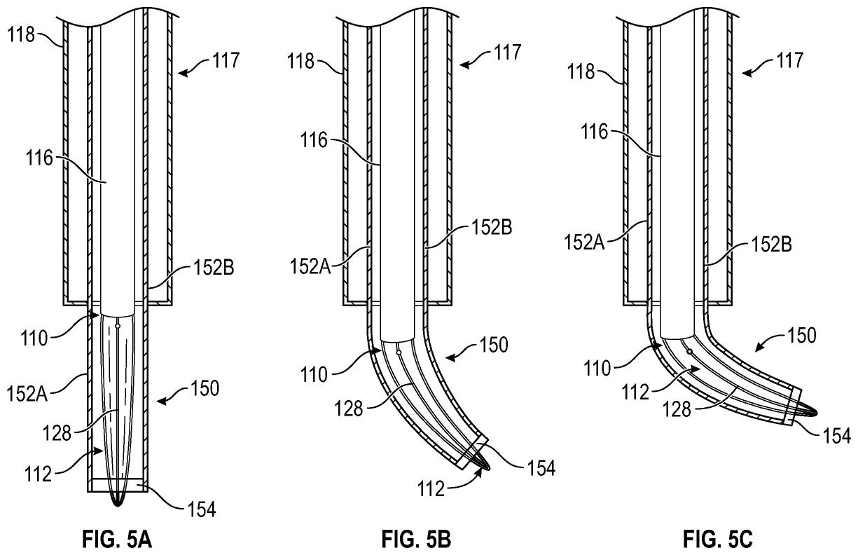

FIGS. 5A-5C show an example of surgical instrument 110 wherein tissue-removal device 112 can be curved relative to central axis A1 of shaft 116 via a separate mechanism, such as before engaging tissue 114. In examples, the separate mechanism can comprise steering device 150. In examples, steering device 150 can comprise extensions 152A and 152B and guide 154. Thus, first component 120A and second component 120B can be configured to be straight at rest, as shown in FIG. 5A, and then curved by advancing and adjusting steering device 150 that can ride along, e.g., on top of, first component 120A and second component 120B. Steering device 150 can be steerable, such as by extensions 152A and 152B comprising or being connected to proximally-controlled pull wires, to shape the radius of curvature of first component 120A and second component 120B to fit the diseased tissue or lesion profile before restationing the tissue. Steering device 150 can be configured similarly to the various examples of brace 140. However, in various examples, extensions can be configured as wires or cables that can be individually tensioned via a control device (e.g., handle section 32 of FIG. 13). Thus, one of extensions 152A and 152B can be reeled in to turn tissue-removal device 112 to the same side of axis A1 that the extension resides.

Guide 154 can include magnetic capabilities to attract first component 120A and second component 120B. Guide 154 can be attached to tissue-removal device 112 via a slotted connection that can permit guide 154 to slide axially along tissue-removal device 112, that can be retracted to allow first component 120A and second component 120B to be at their natural state and advanced to allow first component 120A and second component 120B to be deflected or curved. In examples, extensions 152A and 152B can be connected directly to first component 120A and second component 120B, respectively, without guide 154.

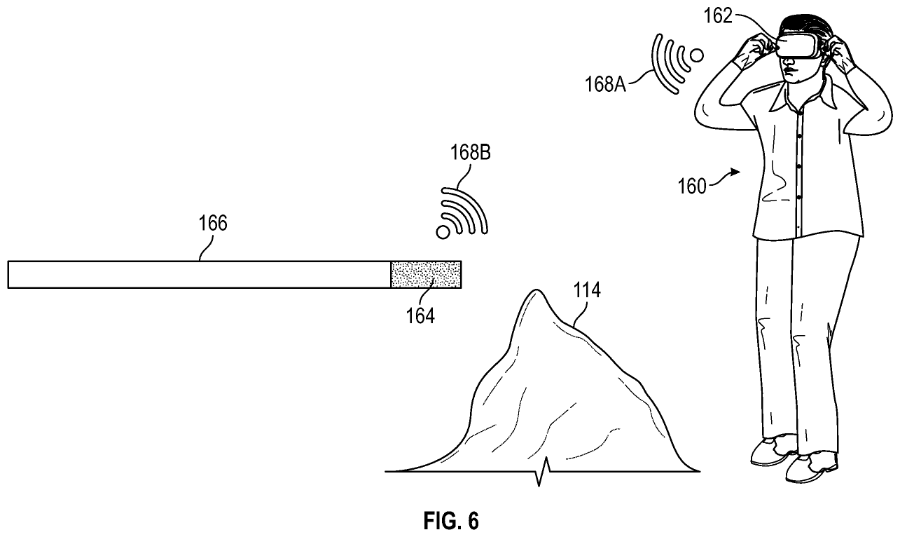

FIG. 6 shows surgeon 160 wearing display device 162 that can communicate with imaging module 164 disposed at the distal end of insertion device 166. Display device 162 can comprise viewing glasses with the following features: 3D imaging, several filter options (NBI, etc.), high resolution (equivalent to 4K) & refresh rates, convenience (light, portable, recharge features, comfort, etc.), compatible lap camera, extremely affordable, vocal switching to the desired feature on a menu option (refer “ok Google” on cell phone). Imaging module 164 can comprise a sealed, self-contain device include various combinations of the following features as well as other features: a camera, a light emitting diode (LED), a battery, a wireless communication device and the like. Imaging module 164 and display device 162 can be configured to communicate via wireless signals 168A and 168B. In examples insertion device 166 can comprise scope 14 of FIGS. 13-15B. In examples, insertion device 166 can be disposable and can attach to an reusable imaging module that can be sealed for washing and sterilizing.

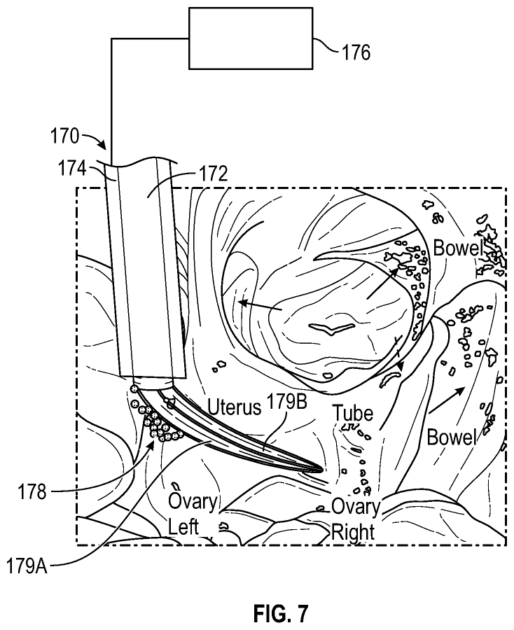

FIG. 7 shows a uterus into which adhesion suppressant device 170 is inserted. Adhesion suppressant can be delivered directly to the resection site using insertion device 172, such as a laparoscope. Adhesion suppressant tube 174 can be connected to or disposed in insertion device 172 and can be fluidly connected to adhesion suppressant canister 176. Adhesion suppressant canister 176 can be fitted into the device handle and delivered to a distal end portion of insertion device 172 via tube 174 or another mechanism. Adhesion suppressant 178 can comprise low viscosity materials for easy flow through a delivery tube to the resection location and that can change to a high viscosity at body temperature (e.g., 34° C.) to reach a gel consistency and adhere in place. Delivery device 172 for the adhesion suppressant can comprise a shaft of combination device including flexible tissue removal devices (e.g., scissors) described herein with adhesion suppressant delivery capability. For example, delivery device 172 can comprise first component 179A and second component 179B, which can be configured similarly as the various examples of first component 120A and 120B described herein. In examples delivery device 172 can comprise scope 14 of FIGS. 13-15B.

Adhesions can form according to a pathophysiology as a natural part of the body's healing process after surgery in a similar way that a scar forms. Adhesion formation post-surgery typically occurs when two injured surfaces are close to one another. This often causes inflammation and causes fibrin deposits onto the damaged tissues. The fibrin then connects the two adjacent structures where damage of the tissues occurred. In 2002, Giuseppe Martucciello's research group (Torre M, Favre A, Pini Prato A, Brizzolara A, Martucciello G (December 2002). “Histologic study of peritoneal adhesions in children and in a rat model”. Pediatr. Surg. Int. 18 (8): 673-6. doi:10.1007/s00383-002-0872-6. PMID 12598961. S2CID 26508386) showed a possible role could be played by microscopic foreign bodies inadvertently contaminating the operative field during surgery. These data suggested that two different stimuli are necessary for adhesion formation: a direct lesion of the mesothelial layers and a solid substrate foreign body. The present disclosure can comprise a material dispensed during resection, to protect the wound from adjacent anatomy pushing against it and interfering with the healing process.

An adhesion suppressant can be a foam-like/gel-like material, with hemostatic/healing/antimicrobial ingredients/properties; that encapsulates the re-sectioned region for some period of time. The purpose is to protect the wound, creating separation from adjacent anatomy and preventing adhesions from forming. The foam/gel biodegrades over a few days, and is absorbed by the body.

Examples of thermoresponse soluble gels suitable for adhesion suppressant delivery, are described in “Formulation, functional evaluation and ex vivo performance of thermoresponsive soluble gels—A platform for therapeutic delivery to mucosa sinus tissue” by Preeti Pandey et al., published in the European Journal of Pharmaceutical Sciences published online 19 Oct. 2016.

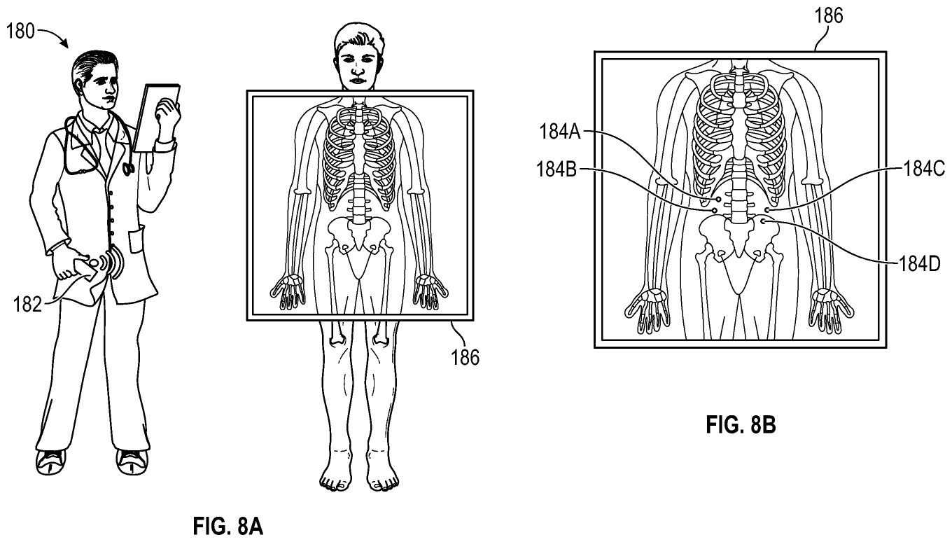

FIGS. 8A and 8B show system 180 for monitoring surgical outcomes. Physicians typically lack ways to measure surgical outcomes. The present disclosure can provide ways to measure surgical outcomes through tactile stimulation of an internal anatomical point and by monitoring inflammation, tissue growth, etc. System 180 can comprise computing device 182 and sensors 184A-184D. Sensors 184A-184D can be viewable in imaging device 186, which can comprise an x-ray machine or another imaging system such as a computerized tomography (CT) system or ultrasound system.

Examples can comprise nano-sized sensors 184A-184D implanted during surgery at each surgical location, and programmed to biodegrade in approximately one to two years, or other time frames. Sensors 184A-184D can be stimulated by an external source, such as computing device 182 emitting a Bluetooth or radiofrequency (RF) field. The stimuli can activate sensors 184A-184D, making each vibrate at the implanted location. The patient can sense the vibration and relate the vibration to the anatomical location. The physician can then be able to complement other diagnostic data with this information and determine the outcome of the surgery.

Examples of ultrasound powered implants suitable for use as in-situ feedback devices are described in “Ultrasound Powered Implants: Design, Performance Considerations and Simulation Results” by Bruno Miguel Gil Rosa et al., published in Scientific Reports by Nature Research on Apr. 16, 2020.

Additional examples can comprise a radio opaque tattoo that can be placed during surgery at each surgical location, and programmed to biodegrade in one to two years, or other time frames. The tattoo can be imaged and movement of the tattoo can be tracked periodically during patient visits. Changes in direction, movement, distance travelled, etc. cab be computed. The physician can then be able to complement other diagnostic data with this information and determine the outcome of the surgery.

FIGS. 9A and 9B are schematic illustrations of surgical instrument 190 comprising flexible electrically conductive cutting, slicing or shaving device 192, which can be configured to bend similarly as is described with reference to the cutting instrument of FIGS. 1A-5C. Cutting device 192 can comprise flexible body 194 and conductor wire 196. Surgical instrument 190 can comprise cutting device 192 and shaft 198. In various examples, flexible electrically conductive cutting device 192 can include tissue-removing edges that can be deformed to fit against or conform to surfaces of tissue to, for example, facilitate flush removal of tissue with an anatomic wall such that scraping or reciprocation of device 192 can remove tissue in the deformed state.

Cutting device 192 of FIGS. 9A and 9B can comprise flexible body 194 made of an insulative material. In examples, flexible body 194 can be fabricated from plastic materials, fiberglass materials, ceramic materials and the like. Flexible body 194 can additionally be reinforced with strengthening elements or biasing elements, such as pre-curved metal wires that are electrically insulated from conducting wire 196, such as by the material of flexible body 194 or wire jacketing. Conductor wire 196 can extend along an edge of flexible body 194 or otherwise be exposed to the exterior of flexible body 194. Flexible body 194 and conductor wire 196 can be connected to a distal end portion of shaft 198. Conductor wire 196 can be fabricated from a conducting material, such as copper or stainless steel, and can be connected to an electrical generator at a proximal end of shaft 198, such as one located in an operating room outside of a patient, including control unit 16 (FIG. 13). The electrical generator can be configured to energize conductor wire 196 to perform cutting of tissue, such as via ablation, cauterization, etc. In examples, conductor wire 196 can additionally include a cutting blade edge. Flexible body 194 can be sufficiently thin to allow flexure and conformance to tissue. Flexible body 194 can be biased or flexed via any of the methods described with reference to FIGS. 1A-5C. For example, flexible body 194 can be biased to straight or curved positions and flexed or straightened via an actuation device, steering device or sleeve. The force exerted to deflect cutting device 192 can be a force suitable for deflecting flexible body 194 and conductor wire 196 without undue exertion from a user, with minimizing risk of puncturing through an anatomy wall and below a level where stresses in strains are generated in flexible body 194 and conductor wire 196 that might give rise to permanent bending or breaking.

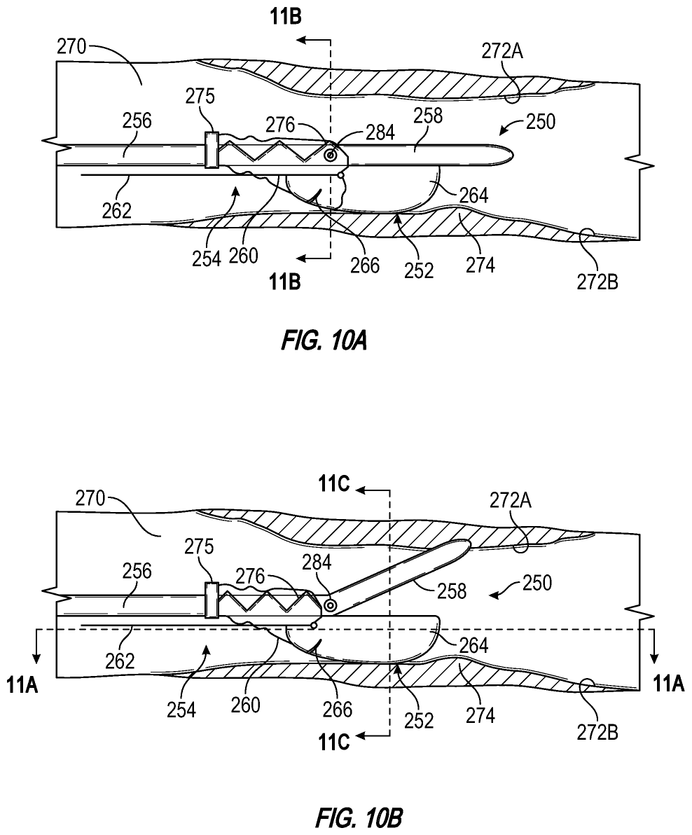

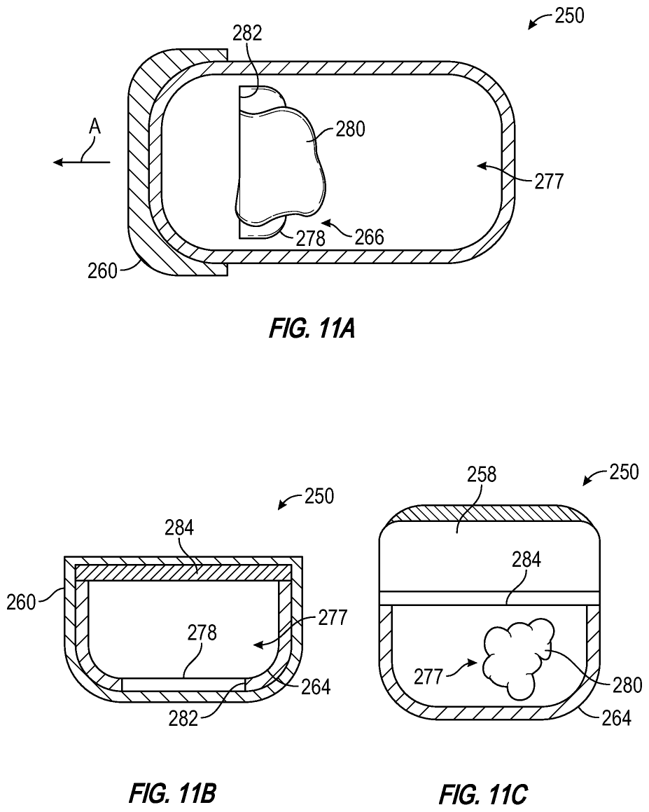

FIGS. 10A-11C are schematic illustrations of a surgical instrument comprising an elongate shaft and a tissue scraping device having a tissue container and a biasing element. The surgical instrument of FIGS. 10A-11C can be configured to bend similarly as is described with reference to the cutting instrument of FIGS. 1A-5C. Thus, one or more of the elongate shaft, tissue scraping device and tissue container can be flexible to facilitate engagement with tissue at different angles, such as via induced bending from the biasing element.

FIG. 10A is a schematic illustration of tissue retrieval device 250 comprising scraping device 252 and spring-loaded actuator 254 in a retracted state. FIG. 10B is a schematic illustration of tissue retrieval device 250 with spring-loaded actuator 254 in a deployed state. In the illustrated example, tissue retrieval device 250 and actuator 254 can be mounted to shaft 256, proximate a distal end of shaft 256. Actuator 254 can comprise projection 258, sleeve 260 and pull-cord 262. Scraping device 250 can comprise container 264 and blade 266.

As shown in FIG. 10A, tissue retrieval device 250 can be positioned in anatomic duct 270, which can comprise first wall portion 272A and second wall portion 272B. Second wall portion 272B can comprise target tissue 274. Shaft 256 can be used to guide scraping device 252 through anatomic duct 270 to target tissue 274. Target tissue 274 can comprise a protrusion, such as a growth of cancerous or pre-cancerous material.

Tissue retrieval device 250 can be inserted into anatomic duct 270 with sleeve 260 in a distal or disengaged position, as shown in FIG. 10A. Sleeve 260 can be retracted proximally using pull-cord 262. Sleeve 260 can be anchored to shaft 256 using band 275, which can be axially fixed relative to shaft 256. Pull-cord 262 can be coupled to a distal end portion of sleeve 260, such as by using a mechanical fastener, a chemical bond or an adhesive, etc. Sleeve 260 can comprise a piece of material that surrounds, or at least partially surrounds tissue retrieval device 250. Sleeve 260 can be pliable so as to be furled between band 275 and the distal end portion of sleeve 260. Sleeve 260 can incorporate, or otherwise be configured to operate with, spring 276, or another biasing element, that can be used to bias the distal end portion of sleeve 260 distally away from band 275. Thus, pull-cord 262 can be drawn proximally to retract sleeve 260 and expose blade 266 and thereby furrow sleeve 260 out of the way of blade 266. Pull-cord 262 can be released so that spring 276 can push the distal end portion of sleeve 260 distally to cover blade 266. In other examples, sleeve 260 and pull-cord 262 can comprise rigid bodies, with band 275 and spring 276 omitted. As such, sleeve 260 can be slid along shaft proximally and distally by pulling and pushing of pull-cord 262.

FIG. 11A is a top cross-sectional view of tissue retrieval device 250 of FIGS. 10A and 6B showing space 277 within container 264 for the storage of collected matter. As shown in FIG. 11A, sleeve 260 can be retracted proximally in the direction of arrow A. For example, pull-cord 262 can be pulled proximally to pull sleeve 260 away from blade 266. With pressure-applying device 250 activated, blade edge 278 can be pressed against target tissue 274 (FIG. 10A). Tissue retrieval device 250 can be moved in the direction of arrow A, such as by pulling on shaft 256 (FIG. 10A) by a user, to cause sample tissue 280 to move into container 264. Tissue retrieval device 250 can be reciprocated back-and-forth along the axis of arrow A to collect additional pieces of sample tissue 280.

FIG. 11B is a side cross-sectional view of tissue retrieval device 250 of FIGS. 10A and 10B showing blade edge 278 of blade 266 for the slicing of biological matter to be collected. Blade 266 can comprise a device configured to simultaneously separate tissue from anatomic duct 270 and direct separated tissue into space 277 of container 264. Blade edge 278 can be fabricated out of an edge of opening 282 in container 264. In examples, blade edge 278 can be curved into or out of container 264 to facilitate engagement with and slicing of target tissue 274 (FIG. 10A). In examples, blade 266 can be configured similarly to a potato peeler, including so-called swivel peelers that can comprise a separate blade edge from container 264 that is mounted on pivot points within an aperture in container 264.

As shown in FIG. 11B, sleeve 260 can be configured to cover blade 266 in a non-retracted or distally-positioned state. As such, tissue retrieval device 250 can be configured to be inserted into a patient, e.g., distally into anatomic duct 270 (in the opposite direction of where arrow A is pointing), without unintentionally engaging anatomic duct 270. Furthermore, in the non-retracted state, sleeve 260 can be configured to push actuator 258 radially inward, relative to the axis of shaft 256, toward container 264. In the illustrated example, sleeve 260 can push actuator 258 against container 264 to thereby retain sample tissue 280 therein.

FIG. 11C is a side cross-sectional view of tissue retrieval device 250 of FIGS. 10A and 10B showing sleeve 260 positioned around actuator 256 and container 264. Actuator 256 can comprise biasing element 284, such as a spring, to cause projection 258 to move away from container 264. Projection 258 can be configured to push scraping device 252 perpendicular to the axis of shaft 256 and additionally cause bending of shaft 256 and scraping device 252. However, due to rotation of actuator 256 about biasing element 284, projection 258 can be configured to provide axial biasing to scraping device 252. The length of projection 258 can be configured to provide a desired amount of pressure or bending for scraping device 252, with longer projections 258 being configured to apply more force and induce more bending. Thus, projection 258 can extend beyond the distal end of container 264. Tissue retrieval device 250 can be configured in multiple models or configurations with different sized (e.g., lengths) projections 258 for use in different sized body cavities.

In examples of the present disclosure, shaft 256 can be configured to flex. Additionally, scraping device 252 can be configured to flex. Shaft 256 and scraping device 252 can be biased or flexed via any of the methods described with reference to FIG. 1A-5C or with spring-loaded actuator 254. For example, shaft 256 and device 252 can be biased to straight or curved positions and flexed or straightened via an actuation device, steering device or sleeve, or spring-loaded actuator 254.

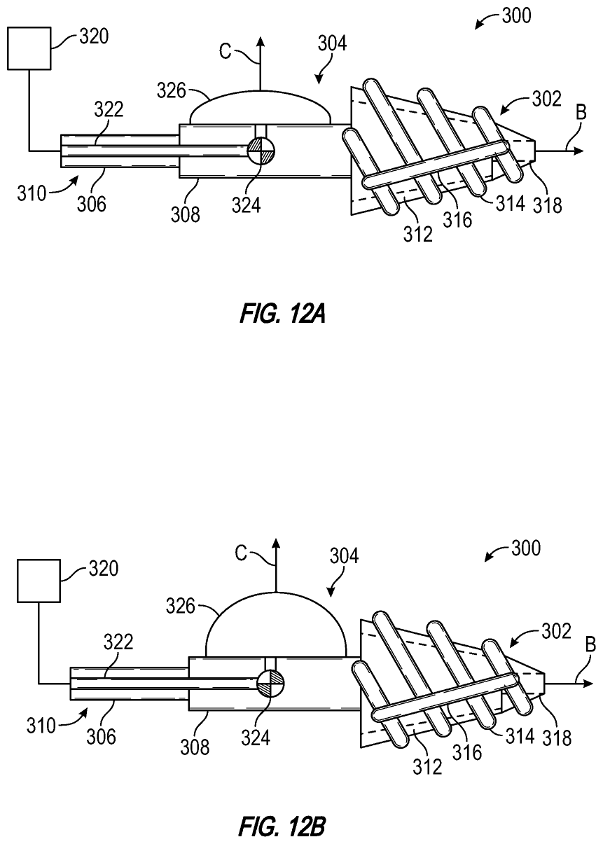

In examples, shaft 256 can be configured to flex either by inclusion of a pre-bend or by inducement of a pre-bend by an activating device. In examples, shaft 256 can be fabricated of shape memory alloy, such as Nitinol or Nickel titanium. In examples, shaft 256 can be fabricated of stainless steel that is deformed into a curved shape. In additional examples, shaft 256 can be fabricated from plastic materials, with or without internal or external reinforcements that can aid in pre-pending. Furthermore, shaft 256 can be induced to bend via brace 140 (FIG. 3A-4B) or steering device 150 (FIGS. 5A-5C), as well as projection 258 and inflatable actuator 304 (FIGS. 12A and 12B).

Container 264 can be fabricated from a flexible material, such as a wire mesh or a woven fabric, to accommodate flexing of scraping device 252. However, container 264 can also be omitted.

In additional examples, tissue retrieval device 250 can comprise a simplified version of scraping device 252. In such configurations, spring-loaded actuator 254 and container 264 can be omitted and scraping device 252 can comprise a thin flexible sheet having blade edge 278 and opening 282 of blade 266 (FIG. 11A) without sidewalls that form container 264. Such examples of scraping device 252 can be readily made to flex via engagement with tissue, by inclusion of a pre-curve or by cooperation with brace 140 (FIG. 3A-4B) or steering device 150 (FIGS. 5A-5C). Tissue retrieval device 250 can be actuated by distal and proximal advancement of blade edge 278. In additional examples, blade edge 278 can be oriented parallel to central axis A1 (FIG. 1A) [e.g., the axis of shaft 256], or closer to parallel (e.g., within forty-five degrees of parallel) to allow shaving or scraping of tissue via rotation of shaft 256.

FIGS. 12A and 12B are schematic illustrations of a surgical instrument comprising an elongate shaft and a tissue boring device having a tissue container and a biasing element. The surgical instrument of FIGS. 12A and 12B can be configured to bend similarly as is described with reference to the cutting instrument of FIGS. 1A-5C. Thus, one or more of the elongate shaft, tissue boring device, tissue container and biasing element can be flexible to facilitate engagement with tissue at different angles, such as via induced bending from the biasing element.

FIG. 12A is a schematic illustration of tissue retrieval device 300 comprising boring device 302 and inflatable actuator 304 in a collapsed state. FIG. 12B is a schematic illustration of tissue retrieval device 300 comprising boring device 302 and inflatable actuator 304 in an expanded state. FIGS. 12A and 12B are discussed concurrently.

Tissue retrieval device 300 can further comprise shaft 306, sleeve 308 and energization system 310. Boring device 302 can comprise container 312, boring lands 314, blade 316 and bore 318. Energization system 310 can comprise energy source 320, duct 322 and valve 324. Actuator 304 can comprise bladder 326.

Tissue retrieval device 300 can be configured to engage tissue in the axial direction of arrow B. For example, tissue retrieval device 300 can be positioned in front of a mound or protrusion of tissue (e.g., target tissue 114 of FIG. 1A) or proximate a wall of tissue (e.g., anatomy wall 130 of FIG. 1A). Shaft 306 can be advanced in the direction of arrow A by a user to engage the target tissue. In some situations, it is possible for boring device 302 to slip over the target tissue, such as due to slippery or moist conditions. Thus, it can be difficult or impossible to engage the tissue sufficiently to collect a desirable volume of sample tissue. Inflatable actuator 304 can be employed to expand in the direction of arrow C, as shown in FIG. 12B, thereby pushing boring device 302 perpendicularly toward the axis of arrow B, and also flexing shaft 306. As such, as boring device 302 is advanced forward, the distal tip of container 312 can maintain engagement with the tissue.

Inflatable actuator 304 can comprise a pressure-applying device configured to be energized with a fluid or gas. Energy source 320 can comprise a source of pressurized fluid, such as air or saline. The pressurized fluid can flow from energy source 320 to bladder 326 through duct 322. Valve 324 can be positioned in duct 322 to selectively allow the pressurized fluid into the internal cavity of bladder 326. Valve 324 can be mechanically or electrically activated and can be controlled by an actuator connected to a handpiece (e.g., handpiece 218 of FIG. 16) located on shaft 306, or an actuator located on control unit 16 (FIG. 16). In other example, valve 324 can be located at a proximal location along shaft 306, such as to be positioned to not be inserted into the patient.

In examples, boring device 302 can be configured as an auger. As such, container 312 can have a cone shape with lands 314 wrapped around container 312 in a spiral manner. Lands 314 can be configured to engage tissue to allow container 312 to penetrate the tissue in the direction of arrow B. Shaft 306 can be rotated by an operator to rotate container 312 and lands 314. Lands 314 can grab tissue while being rotated to cause further axial penetration of boring device 302 into the tissue. Bladder 326 can be mounted on sleeve 308 through which shaft 306 can pass to allow shaft 306 to rotate boring device 302 without affecting the directionality of inflatable actuator 304. As container 312 enters tissue, blade 316 can be configured to slice or shave tissue away from the patient. Blade 316 can comprise a sharpened edge of an opening in container 312 and can be configured similar to a potato peeler as discussed herein. Additionally, container 312 can include distal bore 318 that can be configured to punch through tissue to take a tissue sample similar to core sampling a tree, etc. As such, the distal or leading edge of bore 318 can be sharpened. In examples, only one of blade 316 and bore 318 can be used. In other examples, boring device 302 can be configured to simply punch into the tissue such that tissue enters bore 318. Thus, boring device 302 can be configured as a punch. In such a configuration, lands 314 and blade 316 can be omitted from container 312. In the various examples, container 312 can be configured to have an internal space to capture and retain sample tissue collected by bore 318 and/or blade 316.

In another example, inflatable actuator can be configured to be activated by magnetic repulsion. A first magnet can be attached to bladder 326 and a second magnet can be attached to sleeve 308. Sleeve 308 can be rotated to radially align the first and second magnets to push bladder 326 away from shaft 306. Sleeve 308 can be rotated to un-align the first and second magnets to allow bladder 326 to fall back toward shaft 306. In another example, first and second magnets can be stationarily aligned and can be electromechanically energized to produce a magnetic field.

In examples of the present disclosure, shaft 306 can be configured to flex. Additionally, device 302 and inflatable actuator 304 can be configured to flex. Shaft 306, device 302 and inflatable actuator 304 can be biased or flexed via any of the methods described with reference to FIGS. 1A-5C. For example, shaft 306, device 302 and inflatable actuator 304 can be biased to straight or curved positions and flexed or straightened via an actuation device or sleeve.

In examples, shaft 306 can be configured to flex either by inclusion of a pre-bend or by inducement of a pre-bend by an activating device. In examples, shaft 306 can be fabricated of shape memory alloy, such as Nitinol or Nickel titanium. In examples, shaft 306 can be fabricated of stainless steel that is deformed into a curved shape. In additional examples, shaft 306 can be fabricated from plastic materials, with or without internal or external reinforcements that can aid in pre-pending. Furthermore, shaft 306 can be induced to bend via brace 140 (FIG. 3A-4B) or steering device 150 (FIGS. 5A-5C).

In an example, a tissue-removal device of the present disclosure can comprise a conformable scraping device that can be pushed into engagement with tissue to conform to contours of the tissue. For example, the conformable scraping device can comprise blade 266 (FIG. 11A) without the sidewall portions forming container 264 and bladder 326 (FIG. 12A) attached thereto. As such, bladder 326 can be configured to push a flexible, sheet like scraping or slicing device into close conformance with an anatomic duct wall.

FIG. 13 is a schematic diagram of endoscopy system 10 comprising imaging and control system 12 and endoscope 14. The system of FIG. 13 is an illustrative example of an endoscopy system suitable for use with the systems, devices and methods described herein, such as surgical instrument with flexible end effectors that can be used for obtaining samples of tissue or other biological matter to be removed from a patient for analysis or treatment of the patient. According to some examples, endoscope 14 can comprise scope 117 of FIGS. 1A-5C and can be insertable into an anatomical region for imaging and/or to provide passage of one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises an end-viewing cholangioscope, though other types of endoscopes, such as side-viewing duodenoscopes, can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by pull wires connected to control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as surgical instrument 110 of FIGS. 1A-5C and surgical instrument 190 of FIGS. 9A and 9B. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as ports 40. Knob 38 can be coupled to pull wires, or other actuation mechanisms, extending through insertion section 28 so that rotation of knob 38 can cause bending of functional section 30. Ports 40 can be configured to couple various electrical cables, guide wires, auxiliary scopes, tissue collection devices of the present disclosure, fluid tubes and the like to handle section 32 for coupling with insertion section 28. For example, surgical instrument 110 and surgical instrument 190 can be fed into endoscope 14 via one of ports 40.

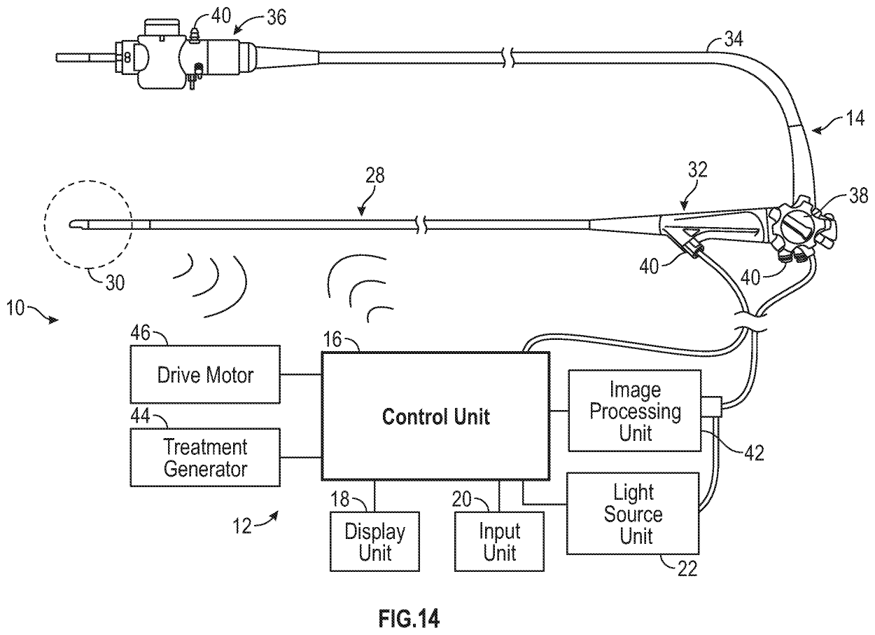

Imaging and control system 12, according to examples, can be provided on a mobile platform (e.g., cart 41) with shelves for housing light source unit 22, suction pump 26, image processing unit 42 (FIG. 14), etc. Alternatively, several components of imaging and control system 12 shown in FIGS. 13 and 14 can be provided directly on endoscope 14 so as to make the endoscope “self-contained.”

Functional section 30 can comprise components for treating and diagnosing anatomy of a patient. Functional section 30 can comprise an imaging device and an illumination device, such as is described further with reference to FIGS. 15A and 15B. Functional section 30 can comprise imaging and illuminating components configured for end-viewing, e.g., viewing distally or axially beyond of functional section 30. In examples, functional section 30 can comprise an elevator as is known in the art of duodenoscopes.

FIG. 14 is a schematic diagram of endoscopy system 10 of FIG. 13 comprising imaging and control system 12 and endoscope 14. FIG. 14 schematically illustrates components of imaging and control system 12 coupled to endoscope 14, which in the illustrated example comprises an end-viewing cholangioscope. Imaging and control system 12 can comprise control unit 16, which can include or be coupled to image processing unit 42, treatment generator 44 and drive unit 46, as well as light source unit 22, input unit 20 and output unit 18.

Image processing unit 42 and light source unit 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 13) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 15A and 15B illustrate an example of functional section 30 of scope 14 of FIG. 14. FIG. 15A illustrates an end view of functional section 30 and FIG. 15B illustrates a cross-sectional view of functional section 30 taken along section plane 15B-15B of FIG. 15A. FIGS. 15A and 15B each illustrate “end-viewing endoscope” (e.g., gastroscope, colonoscope, cholangioscope, etc.) camera module 70. In end-viewing endoscope camera module 70, illumination and imaging systems are positioned such that the viewing angle of the imaging system corresponds to a target anatomy located adjacent an end of endoscope 14 and in line with central longitudinal axis A2 of endoscope 14.

In the example of FIGS. 15A and 15B, end-viewing endoscope camera module 70 can comprise housing 72, therapy unit 74, fluid outlets 76, illumination lens 78 and objective lens 80. Housing 72 can comprise and endcap for insertion section 28, thereby providing a seal to lumen 82.

As can be seen in FIG. 15B, insertion section 28 can comprise lumen 82 through which various components can be extended to connect functional section 30 with handle section 32 (FIG. 14). For example, illumination lens 78 can be connected to light transmitter 84, which can comprise a fiber optic cable or cable bundle extending to light source unit 22 (FIG. 14). Likewise, objective lens 80 can be coupled to imaging unit 87, which can be coupled to wiring 88. Also, fluid outlets 76 can be coupled to fluid lines 89, which can comprise a tube extending to fluid source 24 (FIG. 14). In examples, one of fluid outlets 76 can comprise an inlet connected to a fluid line 89 configured for suction, such as being connected to a vacuum, for recovery of lavage and irrigation fluid. Other elongate elements, e.g., tubes, wires, cables, can extend through lumen 82 to connect functional section 30 with components of endoscopy system 10, such as suction pump 26 (FIG. 14) and treatment generator 44 (FIG. 14). For example, therapy unit 74 can comprise a wide-diameter lumen for receiving other treatment components, such as cutting devices and therapeutic devices including tissue retrieval device 106.

Endoscope camera module 70 can also include a photosensitive element, such as a charge-coupled device (“CCD” sensor) or a complementary metal-oxide semiconductor (“CMOS”) sensor. In either example, imaging unit 87 can be coupled (e.g., via wired or wireless connections) to image processing unit 42 (FIG. 4) to transmit signals from the photosensitive element representing images (e.g., video signals) to image processing unit 42, in turn to be displayed on a display such as output unit 18. In various examples, imaging and control system 12 and imaging unit 87 can be configured to provide outputs at desired resolution (e.g., at least 480p, at least 720p, at least 1080p, at least 4K UHD, etc.) suitable for endoscopy procedures.

FIG. 16 is a schematic illustration of surgical instrument 200 comprising elongate body 202, tissue collection device 204 and device controller 206. Surgical instrument 200 can comprise a device configured for the separation, collection and retrieval of biological matter, such as tissue, from a patient. Tissue collection device 204 can comprise container 210, separator 212, actuation device 214 and activation mechanism 216. Controller 206 can comprise handpiece or handle 218, which can include activation mechanism 216 and connector 220. Elongate body 202 can comprise shaft 222 that can include lumen 224. System controller 206 can be connected to control unit 16 (FIGS. 13 and 14) via cable 226 and the use of connector 220.

Tissue collection device 204 can be configured to do one or both of separate and retrieve biological matter from within a patient after being positioned within the patient by elongate body 202. Tissue collection device 204 can be configured to engage target tissue, aided by operation of actuation device 214, separate the target tissue from the patient and store separated target tissue for removal from the patient, such as by removal of elongate body 202 from the patient.

Handpiece 218 can comprise any device suitable for facilitating manipulation and operation of surgical instrument 200. Handpiece 218 can be located at the proximal end of shaft 222 or another suitable location along shaft 222. In examples, handpiece 218 can comprise a pistol grip, a knob, a handlebar grip and the like. Activation mechanism 216 can be attached to handpiece 218 to operate actuation device 214. Activation mechanism 216 can comprise one or more of buttons, triggers, levers, knobs, dials and the like. Activation mechanism 216 can be coupled to actuation device 214 and can comprise any suitable device for allowing operation of actuation device 214 from handpiece 218. As such, activation mechanism 216 can comprise a linkage located within lumen 224 of shaft 222 or alongside shaft 222. In examples, the linkage can be a mechanical linkage, an electronic linkage or an electric linkage, (such as a wire or cable), or an activation energy source, such as an electric source, a fluid source or a gas source (such as a tube or conduit).

Shaft 222 can extend from handpiece 218 and can comprise an elongate member configured to allow tissue collection device 204 to be inserted into a patient. In examples, shaft 222 can be sized for placement within an auxiliary scope. As such, shaft 222 can be inserted into an incision in the epidermis of a patient, through a body cavity of the patient and into an organ. Thus, it is desirable for the diameter or cross-sectional shape of shaft 222, as well as components attached thereto, to be as small as possible to facilitate minimally invasive surgical procedures. Tissue collection device 204 can thus be incorporated into shaft 222 to minimize the size impact on surgical instrument 200 and without interfering with the linkage. Shaft 222 can be axially rigid, but resiliently bendable, and formed from a metal or plastic material.

Tissue collection device 204 can be located at the distal end of shaft 222 or another suitable location along shaft 222. Tissue collection device 204 can be sized to fit within lumen 119 (FIG. 1A), for example. Tissue collection device 204 can comprise a component or device for interacting with a patient, such as those configured to cut, slice, pull, saw, punch, twist or auger tissue, and the like. Specifically, separator 212 can comprise any device suitable for removing tissue from a patient, such as a blade, punch, scraping device or an auger. In additional examples, separator 212 can comprise a device configured to scrape or abrade tissue from the patient, such as a brush or grater device. In another example, separator 212 can comprise a roughened surface, such as a surface coated with hard particles, such as diamond or sand particles. Separator 212 can be configured to physically separate portions of tissue of a patient from other larger portions of tissue in the patient. In additional examples, separator 212 can be configured to simply collect biological matter from the patient that does not need physical separation, such as mucus or fluid. In examples, separator 212 can be configured to physically separate portion of tissue of a patient for retrieval with the tissue collection device or another device. In examples, tissue collection device 204 can comprise container 210, separator 212, actuation device 214 and activation mechanism 216.

According to the present disclosure, tissue collection device 204 can be configured to bend or flex into different orientations to facilitate engaging tissue along an anatomic wall while reducing the risk of cutting to deeply into the anatomic wall or puncturing the anatomic wall. Tissue collection device 204 can generate curvature under its own power or via an external source, such as from actuation device 214.

Actuation device 214 can comprise a component or system that can be operated to selectively apply curvature to tissue collection device 204, either directly or through shaft 222. Actuation device 214 can be coupled to shaft 222 or tissue collection device 204 in a position to generate force between shaft 222 and tissue collection device 204, thereby causing a change in shape of tissue collection device 204 or shaft 222. In additional examples, actuation device 214 can be positioned to push against biological structure opposite tissue collection device 204 to induce curvature or apply pressure. As such, actuation device 214 can facilitate engagement of separator 212 with target tissue by repositioning, either by direct or indirect curving or by pushing into engagement with tissue. Actuation device 214 can comprise any suitable device for pushing tissue collection device 204. In examples, actuation device 214 can comprise a brace, as described with reference to FIGS. 1A-4B, a steering device, as described with reference to FIGS. 5A-5C, a biasing element, such as a spring-loaded deflector, as is described with reference to FIGS. 10A-11C. In additional examples, actuation device 214 can comprise an inflatable element, such as a balloon or bladder, as is described with reference to FIGS. 12A and 12B. Other pressure-applying forces can be used including magnetic repulsion forces.

Container 210 can be optionally connected to tissue collection device 214 and can be omitted in various examples. Container 210 can comprise a walled element to hold and retain biological matter collected by tissue collection device 204. In an example, container 210 can comprise a flexible basket that can be deformed to allow separator 212 to be brought into close contact with target tissue. For example, container 210 can be fabricated from woven material such as strands of Kevlar, PVC, polyethylene, polycarbonate, PEEK and the like. Container 210 can be coupled to structural components, e.g., a frame, to facilitate coupling to shaft 222 and actuation device 214, as well as to provide stability for separator 212. In additional examples, container 210 can comprise a structural element, such as a box fabricated from rigid and inflexible material.

Handpiece 218 can be operated by a user to operate tissue removal device 204. Handpiece 218 can be used to manipulate shaft 222 to push separator 212 against or angle separator 212 relative to target tissue. For example, shaft 222 can be rotated, oscillated, reciprocated and the like move separator 212 along the target tissue to cause separator 212 to separate sample tissue from the target tissue attached to the patient. Activation mechanism 216 can be coupled to handpiece 218 and can be configured to operate actuation device 214. Activation mechanism 216 can comprise any type of device suitable for activating the different types of actuation devices described herein. In examples, activation mechanism 216 can comprise one or more of a lever, a trigger, a joystick, a button, a wheel and the like, as well as combinations thereof. In an example, activation mechanism 216 can comprise a wheel that can be rotated in one direction to activate or energize an actuation device and rotated in an opposite direction to deactivate or deenergize the actuation device. For example, the wheel can be rotated to push and/or pull a wire or open and close a valve. Activation mechanism 216 can be engaged to activate actuation device such that, for example, the size, geometry or position of actuation device 214 can be changed to induce curvature and/or push against an anatomical surface and cause a corresponding reactive force to be applied to separator 212.

As mentioned, tissue removal device 204 can be configured as a low-profile device so as to be able to be inserted through a small diameter lumen, such as a lumen of an auxiliary scope. Additionally, tissue removal device 204 can be configured as a high-capacity tissue collector that can hold a large volume of collected sample tissue to thereby reduce or eliminate the need to repeatedly remove surgical instrument 200 from the auxiliary scope.

As discussed herein, tissue-removal devices can include incising devices, forceps, scraping devices, slicing devices, cutting devices, electric ablation and cauterizing devices, boring devices and others, and deflecting or bending of tissue-removal devices can be accomplished with pre-biased elements, steering devices, spring-loaded actuators, inflatable devices and others.

### Various Notes & Examples

Example 1 is a surgical instrument for removing biological tissue during a surgical procedure, the instrument comprising: an elongate shaft; and an end effector connected to a distal portion of the elongate shaft, the end effector including an edge configured to remove tissue; wherein curvature of one or both of the elongate shaft and end effector is adjustable to position the edge along a tissue surface when the curvature of the one or both of the elongate shaft and end effector is adjusted.

In Example 2, the subject matter of Example 1 optionally includes wherein the end effector comprises a flexible sheet metal body having a blade edge comprising the edge, the flexible sheet metal body being disposed transverse to a central axis of the elongate shaft.

In Example 3, the subject matter of any one or more of Examples 1-2 optionally include wherein the end effector comprises an electric blade comprising: a flexible insulating body; and a flexible conducting wire extending along the flexible insulating body to define the edge; wherein the flexible conducting wire is connectable to an energization source.

In Example 4, the subject matter of any one or more of Examples 1-3 optionally include wherein one or both of the elongate shaft and end effector are biased to a straight configuration.

In Example 5, the subject matter of any one or more of Examples 1-4 optionally include wherein one or both of the elongate shaft and end effector are biased to a curved configuration.

In Example 6, the subject matter of any one or more of Examples 1-5 optionally include a biasing device to alter the curvature of one or both of the elongate shaft and end effector.

In Example 7, the subject matter of Example 6 optionally includes wherein the biasing device induces the end effector to being straight.

In Example 8, the subject matter of any one or more of Examples 6-7 optionally include wherein the biasing device induces the end effector to being curved.

In Example 9, the subject matter of any one or more of Examples 6-8 optionally include wherein the biasing device comprise a brace slidable along the end effector, the brace comprising: first and second extensions extending alongside the elongate shaft; and a guide connected to the first and second extension and configured to push the end effector to control the curvature.

In Example 10, the subject matter of any one or more of Examples 6-9 optionally include wherein the biasing device comprises a steering device configured to pull the end effector in one or more directions, the steering device comprising: first and second pull wires extending alongside the elongate shaft, each of the first and second pull wires connected to the end effector; and a control device connected to the elongate shaft to adjust tension in the first and second pull wires.

In Example 11, the subject matter of any one or more of Examples 6-10 optionally include wherein the biasing device comprises a spring-loaded actuator comprising: a projection; a hinge connecting the projection to the elongate shaft or the end effector; and a spring to push the projection to alter curvature of the end effector.

In Example 12, the subject matter of any one or more of Examples 6-11 optionally include wherein the biasing device comprises an inflatable device comprising: a bladder mounted to the elongate shaft or the end effector; and a conduit extending along the elongate shaft configured to connect to a pressurization source to provide pressurized fluid to the bladder.

In Example 13, the subject matter of any one or more of Examples 1-12 optionally include wherein means for controlling curvature of one or both of the elongate shaft and end effector comprises a pre-curve introduced into one or both of the elongate shaft and end effector.

Example 14 is a surgical instrument comprising: an elongate shaft extending along a central axis between a proximal end portion and a distal end portion; a control device connected to the proximal end portion; and a tissue-removal device comprising: a flexible body connected to the distal end portion along a trajectory; and a tissue removal feature on the flexible body; wherein a user of the surgical instrument can alter the trajectory of the flexible body by changing curvature of the flexible body.

In Example 15, the subject matter of Example 14 optionally includes wherein the flexible body comprises: a first sheet metal body having a first blade edge; and a second sheet metal body having a second blade edge; wherein the first and second blade edges for the tissue shaving feature.

In Example 16, the subject matter of any one or more of Examples 14-15 optionally include wherein the flexible body comprises a sheet metal body having a shaving window that is disposed along an angle transverse to the trajectory.

In Example 17, the subject matter of any one or more of Examples 14-16 optionally include wherein the flexible body comprises: a flexible insulating body; and a flexible conducting wire extending along an edge of the flexible insulating body; wherein the flexible conducting wire is connectable to an energization source.

In Example 18, the subject matter of any one or more of Examples 14-17 optionally include a device to adjust curvature of the flexible body, the device configured to increase a radial distance between a tip of the tissue-removal device and the central axis.

In Example 19, the subject matter of any one or more of Examples 14-18 optionally include wherein the device to adjust curvature of the flexible body comprises a steerable or pre-curved guide slidably connected to the tissue-removal device.

Example 20 is a method of incising target tissue from an anatomic wall, the method comprising: inserting a shaft of a surgical instrument into an anatomic chamber of a patient; positioning a tissue-removal device connected to the shaft proximate the target tissue; deflecting an axis of the tissue-removal device relative to a central axis of the shaft; and shaving a surface of the anatomic wall by moving the tissue-removal device in a deflected state along the target tissue.

In Example 21, the subject matter of Example 20 optionally includes wherein positioning the tissue-removal device connected to the shaft proximate target tissue comprises positioning a shaft of the axis perpendicular to the target tissue.

In Example 22, the subject matter of any one or more of Examples 20-21 optionally include wherein deflecting an axis of the tissue-removal device relative to a central axis of the shaft comprises position an axis of the tissue-removal device to be oblique to the anatomic wall.

In Example 23, the subject matter of Example 22 optionally includes wherein deflecting an axis of the tissue-removal device relative to a central axis of the shaft comprises position an axis of the tissue-removal device to be parallel to the anatomic wall.

In Example 24, the subject matter of any one or more of Examples 20-23 optionally include wherein deflecting an axis of the tissue-removal device relative to a central axis of the shaft comprises pushing the tissue-removal device against the anatomic wall.

In Example 25, the subject matter of any one or more of Examples 20-24 optionally include wherein deflecting an axis of the tissue-removal device relative to a central axis of the shaft comprises extending the tissue-removal device from a lumen of an insertion device to release a pre-bend of the tissue-removal device.

In Example 26, the subject matter of any one or more of Examples 20-25 optionally include wherein deflecting an axis of the tissue-removal device relative to a central axis of the shaft comprises guiding a pre-curved brace along the tissue-removal device to impart deflection to the tissue-removal device.

In Example 27, the subject matter of any one or more of Examples 20-26 optionally include wherein deflecting an axis of the tissue-removal device relative to a central axis of the shaft comprises steering the tissue-removal device with one or more pull wires.

In Example 28, the subject matter of any one or more of Examples 20-27 optionally include wherein deflecting an axis of the tissue-removal device relative to a central axis of the shaft comprises actuating a deflector to bend the tissue-removal device.

In Example 29, the subject matter of any one or more of Examples 20-28 optionally include wherein deflecting an axis of the tissue-removal device relative to a central axis of the shaft comprises inflating a bladder to bend the tissue-removal device.

In Example 30, the subject matter of any one or more of Examples 20-29 optionally include wherein removing the target tissue from the anatomic wall with the tissue-removal device in a deflected state comprises electrically activating the tissue-removal device to ablate or cauterize tissue.

In Example 31, the subject matter of any one or more of Examples 20-30 optionally include wherein removing the target tissue from the anatomic wall with the tissue-removal device in a deflected state comprises reciprocating or rotating a tissue scraper.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A surgical instrument for removing biological tissue during a surgical procedure, the instrument comprising:
an elongate shaft; and
an end effector connected to a distal portion of the elongate shaft, the end effector including an edge configured to remove tissue;
wherein curvature of one or both of the elongate shaft and end effector is adjustable to position the edge along a tissue surface when the curvature of the one or both of the elongate shaft and end effector is adjusted.

2. The surgical instrument of claim 1, wherein the end effector comprises a flexible sheet metal body having a blade edge comprising the edge, the flexible sheet metal body being disposed transverse to a central axis of the elongate shaft.

3. The surgical instrument of claim 1, wherein the end effector comprises an electric blade comprising:
a flexible insulating body; and
a flexible conducting wire extending along the flexible insulating body to define the edge;
wherein the flexible conducting wire is connectable to an energization source.

4. The surgical instrument of claim 1, wherein one or both of the elongate shaft and end effector are biased to a straight configuration.

5. The surgical instrument of claim 1, wherein one or both of the elongate shaft and end effector are biased to a curved configuration.

6. The surgical instrument of claim 1, further comprising a biasing device to alter the curvature of one or both of the elongate shaft and end effector.

7. The surgical instrument of claim 6, wherein the biasing device induces the end effector to being straight.

8. The surgical instrument of claim 6, wherein the biasing device induces the end effector to being curved.

9. The surgical instrument of claim 6, wherein the biasing device comprise a brace slidable along the end effector, the brace comprising:
first and second extensions extending alongside the elongate shaft; and
a guide connected to the first and second extension and configured to push the end effector to control the curvature.

10. The surgical instrument of claim 6, wherein the biasing device comprises a steering device configured to pull the end effector in one or more directions, the steering device comprising:
first and second pull wires extending alongside the elongate shaft, each of the first and second pull wires connected to the end effector; and
a control device connected to the elongate shaft to adjust tension in the first and second pull wires.

11. The surgical instrument of claim 6, wherein the biasing device comprises a spring-loaded actuator comprising:
a projection;
a hinge connecting the projection to the elongate shaft or the end effector; and
a spring to push the projection to alter curvature of the end effector.

12. The surgical instrument of claim 6, wherein the biasing device comprises an inflatable device comprising:
a bladder mounted to the elongate shaft or the end effector; and
a conduit extending along the elongate shaft configured to connect to a pressurization source to provide pressurized fluid to the bladder.

13. The surgical instrument of claim 1, wherein means for controlling curvature of one or both of the elongate shaft and end effector comprises a pre-curve introduced into one or both of the elongate shaft and end effector.

14. A surgical instrument comprising:
an elongate shaft extending along a central axis between a proximal end portion and a distal end portion;
a control device connected to the proximal end portion; and
a tissue-removal device comprising:
a flexible body connected to the distal end portion along a trajectory; and
a tissue removal feature on the flexible body;
wherein a user of the surgical instrument can alter the trajectory of the flexible body by changing curvature of the flexible body.

15. The surgical instrument of claim 14, wherein the flexible body comprises:
a first sheet metal body having a first blade edge; and
a second sheet metal body having a second blade edge;
wherein the first and second blade edges for the tissue shaving feature.

16. The surgical instrument of claim 14, wherein the flexible body comprises a sheet metal body having a shaving window that is disposed along an angle transverse to the trajectory.

17. The surgical instrument of claim 14, wherein the flexible body comprises:
a flexible insulating body; and
a flexible conducting wire extending along an edge of the flexible insulating body;
wherein the flexible conducting wire is connectable to an energization source.

18. The surgical instrument of claim 14, further comprising a device to adjust curvature of the flexible body, the device configured to increase a radial distance between a tip of the tissue-removal device and the central axis.

19. The surgical instrument of claim 14, wherein the device to adjust curvature of the flexible body comprises a steerable or pre-curved guide slidably connected to the tissue-removal device.
